(19) United States
(12) Patent Application Publication
(10) Pub. No.: US 2016/0008990 A1
(43) Pub. Date: Jan. 14, 2016

(54) MANIPULATION AND/OR EXAMINATION INSTRUMENT

(71) Applicant: Scholly Fiberoptic GmbH, Denzlingen (DE)

(72) Inventors: Marcus Franz, Bad Krozingen-Biengen (DE); Klaus Wolter, Bad Krozingen (DE); Matthias Kratschmer, Freiburg (DE); Jochen Dietrich, Elzach (DE); Claas Muller, Freiburg (DE)

(73) Assignee: SCHOLLY FIBEROPTIC GMBH, Denzlingen (DE)

(21) Appl. No.: 14/792,972

(22) Filed: Jul. 7, 2015

(30) Foreign Application Priority Data
Jul. 9, 2014 (DE) 102014010181.9

(51) Int. Cl. B25J 17/00; B25J 18/00; A61B 1/008

(52) U.S. Cl. CPC B25J 17/00; A61B 1/008; B25J 18/00

(57) ABSTRACT

In the case of a manipulation and/or examination instrument (1) which includes at least two links (2, 3) which are connected in an articulated manner to one another via a double joint (4), wherein a bend of the double joint (4) can be specified with at least two pushing and/or pulling elements (5, 6, 7, 34, 35, 36), whose respective lengths can be adjusted individually, it is proposed to form a pivot spacing (10) of the pivots (8, 9) of the double joint (4) to be longer than a free spacing (11) between the links (2, 3).

### INCORPORATION BY REFERENCE

The following documents are incorporated herein by reference as if fully set forth: German Patent Application No. DE 102014010181.9, filed Jul. 9, 2014.

### BACKGROUND

The invention relates to a manipulation and/or examination instrument having at least two links which are connected in an articulated manner to one another, wherein the links are connected via a double joint which comprises two joint pivots which are arranged at a pivot spacing from one another, wherein the double joint can be adjusted with at least two pushing and/or pulling elements having lengths which can be adjusted independently of one another and which between the links in a straight orientation of the double joint traverse, in each case, a free spacing between the links.

Such manipulation and/or examination instruments can be used e.g. in order to perform working steps and/or to carry out examinations in a cavity which is difficult to access externally. Examples of such a manipulation and/or examination instrument are provided in the form of endoscopes or high-pressure cleaning instruments. The manipulation and/or examination instruments described can also be configured additionally or alternatively for different intended purposes.

In comparison with single joints, the use of double joints offers the advantage that a larger angular range for adjusting the links with respect to one another is available.

### SUMMARY

The object of the invention is provide a control for a double joint which permits a defined adjustment of an orientation of the links with respect to one another.

One or more features of the invention are provided for the purpose of achieving this object. In particular, in the case of a manipulation and/or examination instrument of the type described in the introduction, it is thus provided in accordance with the invention that the pivot spacing is larger than the free spacing of at least one of the at least two pushing and/or pulling elements. In this case, the free spacing can be characterised e.g. in that it is equal to a length of an exposed portion of the respective pushing and/or pulling element allocated to the double joint. The pivot spacing which in accordance with the invention is longer in comparison with the free spacing of at least one of the at least two pushing and/or pulling elements prevents the two links, which are connected to one another via the double joint, from being mutually displaceable transversely with respect to a connecting direction with the length of the pushing and/or pulling elements remaining constant. This eliminates any ambiguity in the allocation of an orientation of the links relative to one another for a specified length of the pushing and/or pulling elements. Therefore, the invention ensures that by adjusting a length of the pushing and/or pulling elements, it is possible to achieve in each case a defined orientation of the links connected via the double joint. Therefore, an adjustment of an orientation of the links with respect to one another via a remote control in the form of the pushing and/or pulling elements is permitted. In one embodiment which is particularly simple in structural terms, provision can be made that the pushing and/or pulling elements are each designed as a cable pull.

Preferably, the double joint is designed as a component of a flexible part of the manipulation and/or examination instrument.

In the case of one embodiment of the invention, provision can be made that the double joint can be adjusted with at least three pushing and/or pulling elements having lengths which can be adjusted independently of one another. It is advantageous that an orientation of the mutually connected links relative to one another can be established within a solid angle by selecting the adjustable lengths.

In the case of one embodiment of the invention, provision can be made that the pivot spacing is larger than the free spacing of each pushing and/or pulling element, with which the double joint can be adjusted. It is advantageous that an adjustability of the links with respect to one another whilst the length of the pushing and/or pulling elements remains constant can be prevented by rotating the links with respect to one another about a direction or axis extending in parallel with a longitudinal axis of the manipulation and/or examination instrument. Therefore, a desired angle between the links can be specified unequivocally by specifying associated lengths of the pushing and/or pulling elements.

In the case of one embodiment of the invention, provision can be made that the free spacing of each pushing and/or pulling element is provided by a spacing between a fastening or exit point of the pushing and/or pulling element on one of the two links and an insertion point for the pushing and/or pulling element into a guide channel, e.g. a cable pull guide, on the other one of the two links. It is advantageous that the fastening or exit point and the insertion point each define locations, at which the respective pushing and/or pulling element is bent during adjustment of the orientation of the links with respect to one another. Therefore, the pushing and/or pulling element acts like a bar, which is connected in an articulated manner to each link, of the length of the free spacing of the pushing and/or pulling element. The free spacing can also be characterised as a spacing between the links when the double joint is oriented in a straight or elongated manner, in particular as a spacing at the height of the respective pushing and/or pulling element.

In the case of one embodiment of the invention, provision can be made that the double joint passes through a base surface which is described by fastening or exit points or by insertion points of the at least two pushing and/or pulling elements. Therefore, it is possible in a simple manner for the double joint to be arranged within a mathematical-geometric body which is described by the pushing and/or pulling elements. In this manner, the double joint can be bent in any direction by shortening or lengthening the respective pushing and/or pulling elements.

In the case of one embodiment of the invention, provision can be made that the pushing and/or pulling elements of a double joint have corresponding free spacings in a straight orientation of the double joint. This provides symmetry in relation to the actuation of the pushing and/or pulling elements which simplifies an adjustment of an orientation of the links which are connected to one another by means of the double joint. It is particularly favourable if the pushing and/or pulling elements describe a geometric body, into the interior of which the double joint extends. This has proven to be a particularly stable configuration for preventing undesired adjustments of the links with respect to one another.

In general, for a stable configuration it is favourable if the pushing and/or pulling elements are arranged radially in relation to a longitudinal axis, which is provided by a connecting line of the joint pivots, in an offset manner with respect to the double joint and include the double joint radially therebetween.

In the case of one embodiment of the invention, provision can be made that the double joint is formed from two ball joints, the joint heads of which are connected to one another. Ball joints have the advantage that an adjustability in all directions is permitted. In this case, provision can be made that the joint heads are connected in a rigid manner to one another. This produces a structure which is particularly simple in mechanical terms. As an alternative or in addition, provision can be made that the ball joints are connected to one another via an intermediate piece. It is advantageous that a spacing between the joint heads can be adjusted by means of the intermediate piece. Preferably, the intermediate piece is formed in one piece with the joint heads.

In the case of one embodiment of the invention, provision can be made that the ball joints each define a ball radius and that the joint spacing is longer than the free spacing by a value between 1/4 and 1/6 of the ball radius. It has been proven that particularly effective control characteristics can be achieved in this range. It is particularly favourable if the joint spacing is longer than the free spacing by approximately or precisely 1/5 of the ball radius. In this case, the embodiment of the joint spacing which is longer by 1/5 of the ball radius can be characterised e.g. in that a value of 1/5 is achieved taking into consideration measuring inaccuracies and/or manufacturing tolerances. Particularly favourable control characteristics are achieved for this value. The length difference of 1/5 of the ball radius can be achieved e.g. by virtue of the fact that the joint heads are countersunk with their joint pivots by 1/10 of the ball radius into the respective link.

In the case of one embodiment of the invention, provision can be made that in a straight orientation of the double joint, the joint pivots are arranged symmetrically in relation to a center between the associated links. It is advantageous that the two joints are formed and arranged symmetrically in relation to the links. This simplifies the control characteristics of the double joint.

In the case of one embodiment of the invention, provision can be made that in a straight orientation of the double joint, the free spacings are arranged axially between the joint pivots. This permits an easily controllable actuation of the double joint.

In the case of one embodiment of the invention, provision can be made that a continuous working channel is formed in the links and/or in an intermediate piece connecting the links, e.g. the aforementioned intermediate piece, of the double joint. Preferably, the continuous working channel is formed continuously between a proximal end and a distal end of the manipulation and/or examination instrument. Therefore, the working channel can extend in the links, the joint sockets, the joint heads and the intermediate piece. This working channel can be provided and formed in each case at a distal end e.g. for receiving an endoscope for examinations of the cavity, for transmitting data with an electrical connection line and/or optical signals with an optical wave guide and/or as a liquid channel for conveying a cleaning liquid and/or for receiving an optical wave guide e.g. for illumination purposes. The said components and functional units can thus be arranged in the working channel, preferably in such a manner as to be removable. It is advantageous that functional groups can be provided and/or used for examination and/or manipulation in cavities which are difficult to access externally.

In the case of one embodiment of the invention, provision can be made that the instrument comprises a multiplicity of links which are connected to one another in each case in pairs by means of a double joint, wherein pushing and/or pulling elements, with which a selected double joint cannot be adjusted, is guided in a sliding manner out of a guide channel at the height of this double joint on one of the links and is guided in a sliding manner into a guide channel on the other one of the links. It is advantageous that pushing and/or pulling elements which are not provided for controlling a double joint in question can be guided past this double joint and can be guided to the double joint which is allocated to these pushing and/or pulling elements.

Therefore, a chain of links can be formed, preferably as the aforementioned flexible part, which links are each connected in an articulated manner to one another in pairs, wherein the double joints can be adjusted by means of pushing and/or pulling elements individually, i.e. separately from one another.

In the case of one embodiment of the invention, provision can be made that an actuating apparatus for adjusting the lengths of the pushing and/or pulling elements individually and/or in groups is formed at a proximal end of the instrument. It is advantageous that an orientation and/or a spatial progression of the manipulation and/or examination instrument can be adjusted remotely in a simple manner.

### BRIEF DESCRIPTION OF THE DRAWINGS

The invention will now be explained in greater detail with reference to exemplified embodiments but it is not limited to these exemplified embodiments. Further exemplified embodiments are produced from a combination of the features of individual or several claims for protection with one another and/or with individual or several features of the exemplified embodiment.

In the drawings:

FIG. 1 shows a greatly simplified view of a manipulation and/or examination instrument in accordance with the invention,

FIG. 2 shows a section from a chain of links of the instrument of FIG. 1,

FIG. 3 shows a view in the axial direction of a link of the chain of links as shown in Figure

FIG. 4 shows a sectional view of a double joint along a longitudinal axis of the inventive link chain specified by the double joint, and

FIG. 5 shows an ambiguity in the orientation of links, which are connected in an articulated manner to one another, in the event that all of the adjusted lengths are formed to be of equal length and the length of the joint spacing is not in accordance with the invention.

### DETAILED DESCRIPTION OF THE PREFERRED EMBODIMENT

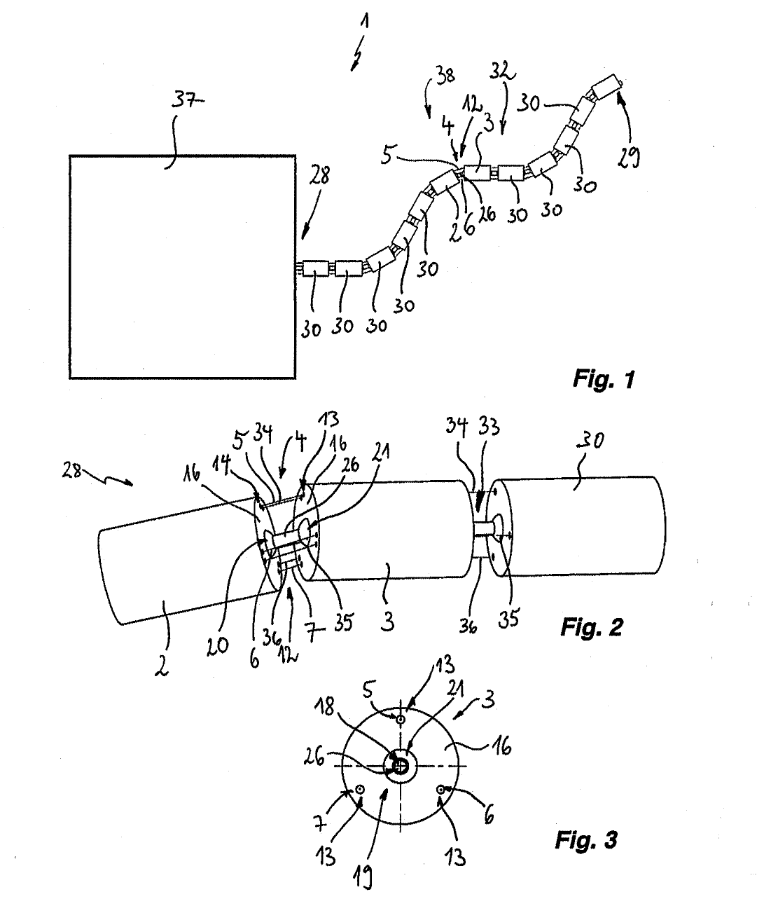

FIG. 1 shows a greatly simplified basic view of a manipulation and/or examination instrument designated on the whole by the reference numeral 1. The manipulation and/or examination instrument 1 has two links 2, 3 which are connected in an articulated manner to one another via a double joint 4.

The double joint 4 permits bending of the link 2 relative to the link 3.

In order to adjust this bend, the manipulation and/or examination instrument 1 has three pushing and/or pulling elements 5, 6, 7 which are designed in this case as cable pulls and whose length can be adjusted individually in each case independently of one another.

FIG. 2 shows by way of example a bent orientation of the link 2 in relation to the link 3, for which the length of the pushing and/or pulling element 7 is shortened with respect to the length of the pushing and/or pulling element 6.

In the sectional view of FIG. 4, it is apparent that the double joint 4 comprises two joint pivots 8, 9 which are arranged spaced apart from one another at a pivot spacing 10.

The link 2 is thus pivotable in its own right about the joint pivot 8, whereas the link 3 is pivotable in its own right about the joint pivot 9.

The function of the double joint 4 is produced in this manner.

FIG. 4 shows the links 2, 3 in a straight orientation, in which the link 3 is arranged flush with the link 2.

In the illustration of FIG. 4, it is apparent that the pushing and/or pulling element 5 bridges the gap 12 between the link 2 and the link 3, which is connected in an articulated manner thereto, via a free spacing 11. In an elongated orientation of the double joint 4, the free spacing 11 is equal to the spacing between the links 2 and 3.

In this case, the joint pivots 8, 9 are arranged on the links 2, 3 in such a manner that the pivot spacing 10 is larger than the free spacing 11 of the pushing and/or pulling element 5.

In a similar manner, the free spacings which are traversed by the pushing and/or pulling elements 6, 7 in the case of a straight orientation of the links 2, 3 with respect to one another through the gap 12 are shorter than the pivot spacing 10.

The pivot spacing 10 is thus larger than the free spacing 11 of each pushing and/or pulling element 5, 6, 7, with which the respective double joint 4 can be adjusted.

In the sectional view in FIG. 4, it is apparent that the pushing and/or pulling element 5 is fastened to the link 3 at a fastening or exit point 13 so that the link 3 can be moved by pulling and pushing on the pushing and/or pulling element 5.

The pushing and/or pulling element 5 is introduced opposite the fastening or exit point 13 at an insertion point 14 into a guide channel 15 which in this case is designed as a cable pull guide.

The fastening or exit point 13 and the insertion point 14 are thus formed in each case on an end surface 16 of the links 2, 3. The end surfaces 16 include the aforementioned gap 12 therebetween and define a boundary of the free spacing 11.

The fastening or exit point 13 on the end surface 16 of the link 3 can also be formed by virtue of the fact that a fastening arrangement is not formed at the height of the end surface 16 but rather further inwards in the guide channel 17, which is designed in this case as a cable pull guide, or at the other end thereof. In this case, the pushing and/or pulling element 5 is also arranged at the fastening or exit point 13 in an invariable manner with respect to the link 3 and during adjustment of the double joint 4 is bent at this location.

In FIG. 4 it is apparent that the free spacing 11 is formed specifically by the spacing between the fastening or exit point 13 and the insertion point 14 in the case of a straight orientation of the double joint 4.

In the illustration of FIG. 3 showing a view along a longitudinal axis 18 of the elongated double joint 4, three fastening or exit points 13 are apparent which are each allocated to a pushing and/or pulling element 5, 6, 7 in the manner described with respect to FIG. 4.

These three fastening or exit points 13 span a triangular base surface which lies in the end surface 16 of the link 3.

In FIG. 4 it is apparent that the double joint 4 passes through this mathematical-geometric base surface which in mathematical terms forms a base surface of a prism which is described by the free spacings 11 of the pushing and/or pulling elements 5, 6, 7 in the case of an elongated double joint 4.

In a similar manner, a similar triangular base surface through which the double joint 4 passes is formed on the end surface 16 of the link 2 by the insertion points 14 of the pushing and/or pulling elements 5, 6, 7.

As a result, the joint pivots 8, 9 are arranged on sides, remote from the gap 12, in relation to the base surfaces 19 or the end surface 16.

The double joint 4 is formed from two ball joints 20, 21 with in each case one joint head 22, 23. In this case, the joint heads 22, 23 are mounted in suitable joint sockets 24, 25.

The joint heads 24, 25 are connected to one another in a rigid manner by means of an intermediate piece 26. The pushing and/or pulling elements 5, 6, 7 thus receive the double joint 4 with the intermediate piece 26 and the joint heads 22, 23 in a radial manner therebetween and surround it in a radial manner outwards.

In the case of the illustrated exemplified embodiment, the free spacings 11 of each pushing and/or pulling element 5, 6, 7 are formed into the double joint 4 having corresponding lengths, if the links 2, 3 are elongated with respect to one another or are oriented in a flush manner.

In this case, the pivot spacing 10 is larger than the free spacing 11 by 1/5 of the ball radius which is defined in each case by the joint heads 22, 23. This value of offsetting the joint pivots 8, 9 back into the links 2, 3 has proven successful for favourable control characteristics. Effective control characteristics are already achieved in the region of a pivot spacing 10, which is larger by 1/4 to 1/6 of the aforementioned ball radius, in comparison with the free spacing 11.

FIG. 4 shows that in a straight or elongated orientation of the double joint 4, the joint pivots 8, 9 are arranged symmetrically in relation to a centre between the end surfaces 16 and thus between the links 2, 3. Therefore, in this orientation of the double joint 4, the free spacings 11 are arranged axially completely between the joint pivots 8, 9.

Formed in the intermediate piece 26, in the joint heads 22, 23 and in the links 2, 3 is a completely continuous working channel 27 which extends between a proximal end 28 and a distal end 29 of the manipulation and/or examination instrument 1. This working channel 27 is arranged e.g. for conveying a working liquid and/or for transmitting electrical and/or optical conductors.

Therefore, a working medium, e.g. a working liquid, light, electrical or optical signals and/or instruments can be moved to the distal end 29 of the manipulation and/or examination instrument or can be guided back therefrom to the proximal end 28.

It is apparent in FIG. 1 that the manipulation and/or examination instrument 1 comprises a multiplicity of links 30 which are each connected in an articulated manner to one another in pairs. These links 30 are each constructed in an identical manner to the links 2, 3 and are connected in an articulated manner to one another in the manner described by double joints. Therefore, a link chain 32 is formed which extends between the proximal end 28 and the distal end 29. In this case, each pair of links 2, 3, 30 connected in an articulated manner to one another is allocated a connecting double joint 33 which is formed in an identical manner to the double joint 4. The links 2, 3, 30 thus form with the double joint 4 a flexible part 38, in particular a flexible probe part, of the manipulation and/or examination instrument 1.

Each double joint 4, 33 is allocated a separate set of pushing and/or pulling elements 5, 6, 7, 34, 35, 36 which in this case are each designed as cable pulls.

For example, the double joint 33 in FIG. 2 is thus allocated the pushing and/or pulling elements 34, 35, 36 which are fastened to the link 30 in the manner described with respect to the link 3 and which are introduced, in the manner described with respect to the link 2, via insertion points in guide channels, which in this case are designed as cable pull guides.

The pushing and/or pulling elements 5, 6, 7, 34, 35, 36 are each guided to an operating unit, with which the length of the pushing and/or pulling elements 5, 6, 7, 34, 35, 36 can be adjusted in each case individually.

Therefore, the pushing and/or pulling elements 34, 35, 36 are also guided via the gap 12 between the link 2 and the link 3, in order to be actuated by the proximal end 28.

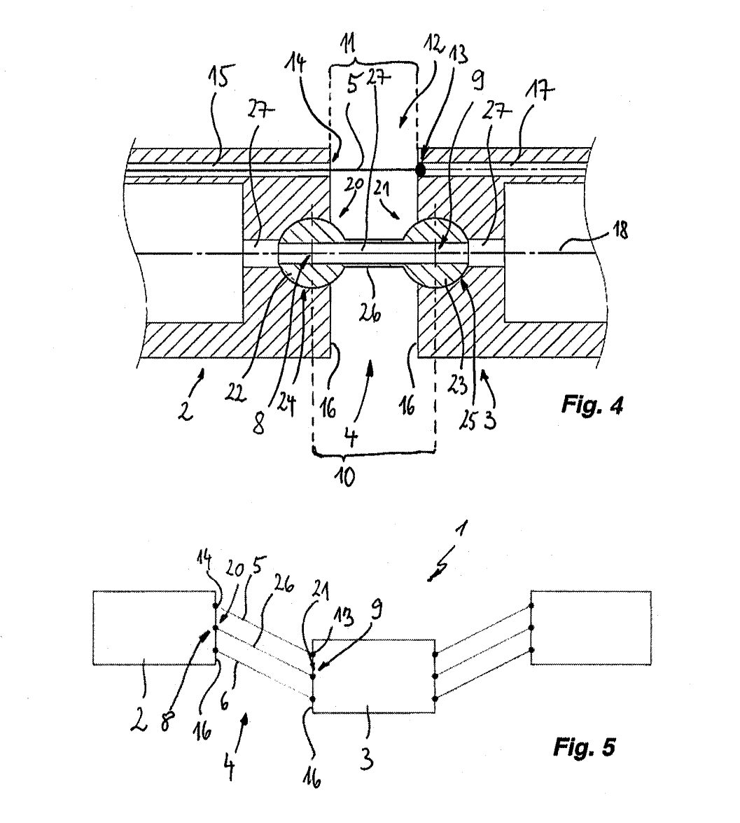

FIG. 5 shows a manipulation and/or examination instrument designated on the whole by the reference numeral 1, wherein the pivot spacing between the joint pivots 8, 9 is not larger than the free spacing 11 between the fastening or exit point 13 and the insertion point 14 and therefore is not in accordance with the invention, but rather wherein these two lengths are precisely the same. It is readily apparent that the links 2, 3 can be displaced relative to one another and in parallel with the end surfaces 16 with lengths of the pushing and/or pulling elements 5, 6 remaining constant. This causes an ambiguity which makes it more difficult to adjust the bend of the double joint 4 via the lengths of pushing and/or pulling elements 5, 6.

In the case of the manipulation and/or examination instrument 1 which comprises at least two links 2, 3 which are connected in an articulated manner to one another via a double joint 4, wherein a bend of the double joint 4 can be specified with at least two pushing and/or pulling elements 5, 6, 7, 34, 35, 36, whose respective lengths can be adjusted individually, it is proposed to form a pivot spacing 10 of the pivots 8, 9 of the double joint 4 to be longer than a free spacing 11 between the links 2, 3.

1. A manipulation and/or examination instrument (1) comprising at least two links (2, 3, 30) which are connected in an articulated manner to one another, a double joint (4, 33) that connects the links (2, 3, 30), the double joint (4, 33) comprises two joint pivots (8, 9) which are arranged at a pivot spacing (10) from one another, at least two pushing and/or pulling elements (5, 6, 7, 34, 35, 36) for adjusting the double joint (4, 33), the at least two pushing and/or pulling elements (5, 6, 7, 34, 35, 36) having lengths which can be adjusted independently of one another and which between the links (2, 3) in a straight orientation of the double joint (4, 33) traverse in each case a free spacing (11) between the links (2, 3, 30), wherein the pivot spacing (10) is larger than the free spacing (11) of at least one of the pushing and/or pulling elements (5, 6, 7, 34, 35, 36) of the at least two pushing and/or pulling elements (5, 6, 7, 34, 35, 36).

2. The manipulation and/or examination instrument (1) as claimed in claim 1, wherein the double joint (4, 33) is adjustable with at least three of the pushing and/or pulling elements (5, 6, 7, 34, 35, 36) having lengths which can be adjusted independently of one another.

3. The manipulation and/or examination instrument (1) as claimed in claim 1, wherein the pivot spacing (10) is larger than the free spacing (11) of each pushing and/or pulling element (5, 6, 7, 34, 35, 36), with which the double joint (4, 33) can be adjusted.

4. The manipulation and/or examination instrument as claimed in claim 1, wherein the free spacing (11) of each pushing and/or pulling element (5, 6, 7, 34, 35, 36) is provided by a spacing between a fastening or exit point (13) of the pushing and/or pulling element (5, 6, 7, 34, 35) on one of the two links (2, 3, 30) and an insertion point (14) for the pushing and/or pulling element (5, 6, 7, 34, 35, 36) into a guide channel (15, 17) on the other one of the two links (2, 3, 30).

5. The manipulation and/or examination instrument (1) as claimed in claim 1, wherein the double joint (4, 33) passes through a base surface (16) which is described by fastening or exit points (13) or by insertion points (14) of the at least two pushing and/or pulling elements (5, 6, 7, 34, 35, 36).

6. The manipulation and/or examination instrument (1) as claimed in claim 1, wherein the pushing and/or pulling elements (5, 6, 7, 34, 35, 36) of the double joint (4, 33) comprise corresponding free spacings (11) in a straight orientation of the double joint (4, 33).

7. The manipulation and/or examination instrument (1) as claimed in claim 1, wherein the double joint (4, 33) is formed from two ball joints, of which, respective joint heads (22, 23) thereof are connected to one another in a rigid manner or by an intermediate piece (26).

8. The manipulation and/or examination instrument (1) as claimed in 7, wherein the ball joints each define a ball radius, and the pivot spacing (10) is longer than the free spacing (11) by a value between 1/4 and 1/6 of the ball radius.

9. The manipulation and/or examination instrument (1) as claimed in claim 1, wherein in a straight orientation of the double joint (4, 33), the joint pivots (8, 9) are arranged symmetrically in relation to a center between the associated links (2, 3, 30) or in a straight orientation of the double joint (4, 33), the free spacings (11) are arranged axially between the joint pivots (8, 9).

10. The manipulation and/or examination instrument (1) as claimed in claim 1, wherein a continuous working channel (27) is formed in at least one of the links (2, 3, 30) or in an intermediate piece (26) of the double joint (4, 33) connecting the links (2, 3, 30).

11. The manipulation and/or examination instrument (1) as claimed in claim 1, wherein the manipulation and/or examination instrument (1) comprises a multiplicity of the links (2, 3, 30) which are connected to one another in each case in pairs by one of the double joints (4, 33), the pushing and/or pulling elements (5, 6, 7, 34, 35, 36), with which a selected one of the double joints (4, 33) cannot be adjusted, are guided in a sliding manner out of a guide channel (15, 17) at a height of said double joint (4, 33) on one of the links (2, 3, 30) and are guided in a sliding manner into a guide channel (15, 17) on the other one of the links (2, 3, 30).

12. The manipulation and/or examination instrument (1) as claimed in claim 1, wherein an actuating apparatus (37) for adjusting the lengths of the pushing and/or pulling elements (5, 6, 7, 34, 35, 36) individually is formed at a proximal end (28) of the instrument (1).
